(19) United States
(12) Patent Application Publication
(10) Pub. No.: US 2012/0022009 A1
(43) Pub. Date: Jan. 26, 2012

(54) TANNATE DRY POWDER FORMULATIONS

(75) Inventor: Thomas Jeffrey Bryant, Winder, GA (US)

(73) Assignee: Acella Pharmaceuticals, LLC

(21) Appl. No.: 13/186,669

(22) Filed: Jul. 20, 2011

Related U.S. Application Data
(60) Provisional application No. 61/366,348, filed on Jul. 21, 2010.

(51) Int. Cl. A61K 31/7028; A61P 37/08

(52) U.S. Cl. 514/25

(57) ABSTRACT

Dry powder tannate compositions containing bioactive agents, tannic acid, dispersants, and viscosity modifying agents are disclosed. Specifically, the bioactive agents are antihistamines, decongestants, antitussives, and anticholinergics. The dry powder formulations can further include pharmaceutically acceptable excipients. The dry powder formulations exhibit increased stability for extended shelf life. Bioactive agent tannate salts remain suspended for at least two weeks following formation of the suspension in a pharmaceutically acceptable aqueous liquid.

### CROSS-REFERENCE TO RELATED APPLICATIONS

This application claims priority under 35 U.S.C. 119 to U.S. Ser. No. 61/366,348 “Tannate Dry Powder Formulations” filed Jul. 21, 2010 by Thomas Jeffrey Bryant, the contents of which are incorporated by reference in their entirety.

### FIELD OF THE INVENTION

The present invention generally relates to tannate powder formulations containing bioactive agents, tannic acid, dispersing agents, and viscosity modifying agents, and methods of making and using thereof.

### BACKGROUND OF THE INVENTION

Due to the hydrophobicity of tannic acid-drug complexes, tannate salts derived from the reaction of a bioactive agent with tannic acid have decreased solubility in aqueous solutions compared to the free base or other salt forms of the bioactive agent. The relatively poor solubility of the tannate salt of the bioactive agent can make it suitable for sustained release over prolonged periods of time. Extended or sustained released formulations can improve patient compliance by reducing the number of required doses and providing therapeutically effective amounts of the bioactive agents over an extended period of time.

Phenylephrine tannate compositions are used as decongestants alone or in combination with antihistamines such as chlorpheniramine tannate and pyrilamine tannate. Solid dosage phenylephrine tannate compositions are described in U.S. Pat. No. 5,599,846. Solid phenylephrine tannate and chlorpheniramine tannate combination compositions are described in U.S. Pat. No. 6,037,358. U.S. Patent Application No. 2008/0125453 describes suspensions additionally including an antitussive.

U.S. Patent Application No. 2005/0202080 describes a wet granulation process for preparing solid dosage formulations containing tannate salts of bioactive agents.

However, degradation of both dry powder bioactive agent tannate blend formulations and bioactive agent tannate suspension formulations can occur. Factors which contribute to the instability of tannate compositions include temperature fluctuation and stress. Many antihistamine tannate compounds undergo decomposition when exposed to prolonged temperatures at or above 50° C. Therefore, preparations requiring heat may lead to degradation and impurities, thereby requiring additional purification steps to comply with quality control guidelines to ensure that consistent therapeutic levels of bioactive agent are present in the dosage formulations. Furthermore, it is known in the art that traditionally prepared liquid suspensions and moist blends of tannate salts readily oxidize upon standing at ambient temperatures for extended periods of time, such as during storage, leading to discoloration, degradation, and/or formation of impurities.

U.S. Pat. Nos. 5,663,415; 5,599,846; and 7,001,886 describe an “isopropanol” route for the preparation of antihistamine tannate solid dosage formulations, which involve a bioactive agent tannate suspension intermediate. However, the yield of this process is often unsatisfactory by industrial manufacturing standards, generally only around 70%, including 2-5% by weight that corresponds to decomposition products which cannot be removed. The purity of these compositions is generally between 85-90%, even though the intermediate suspension is used relatively immediately, i.e. within twelve to twenty-four hours of being generated.

Three general methods of preparing suspension formulations of bioactive tannate salts are described in the art. In the first method, described in U.S. Pat. No. 6,287,597, tannate salts of bioactive agents and accompanying excipients are suspended in a pharmaceutically acceptable liquid. In the second method, described in U.S. Pat. No. 7,273,623, bioactive agents and tannic acid, and optionally an anti-clumping agent, are reacted in the presence of a pharmaceutically active liquid, spontaneously forming the tannate salt of the bioactive agent. In the third method, described in U.S. Application No. 2005/0020509, a pharmaceutically acceptable liquid containing the salt or free base of a bioactive active ingredient is added to a pharmaceutically acceptable liquid containing a dispersing agent and tannic acid.

Dispersing agents are used in suspension formulations to prevent clumping. The dispersing agents used in the bioactive agent tannate suspensions described above are magnesium aluminum silicate and xantham gum. Xantham gum is used as a dispersing agent in the commercial manufacturing process of either suspension dosage forms or transiently formed suspensions prepared during the manufacture of solid dosage forms. However, in order to disperse a suspension effectively without the formation of clumps, formulations using xantham gum as a dispersant require the application of high shear. A number of bioactive agents are sensitive to shear stress, and the use of high speed mixing equipment can lead to degradation. In addition, formulations including bioactive agents, tannic acid, and metals, present in dispersants such as magnesium aluminum silicate, are capable of forming an insoluble complex that renders the bioactive agent insoluble and therefore unable to be efficiently absorbed by the body. Finally, metal ions can catalyze reactions, such as oxidation or hydrolysis reactions, which degrade the active agents. None of the above-mentioned suspension formulations include a discussion of stability or shelf-life upon long-term storage.

There is a need for dry powder formulations containing bioactive agents and tannic acid with extended shelf-lives that do not undergo chemical degradation due to oxidation, stress during manufacture, temperature variations, and/or equilibration between tannate and other salt forms upon suspension. There is a further need for stable bioactive agent tannate suspension formulations derived from stable dry powder formulations.

It is therefore an object of the present invention to provide stable dry powder formulations of tannic acid, bioactive agents, dispersing agents, and viscosity modifying agents with improved shelf-life. The dry powder formulations should be stable for two weeks to 12 months, more preferably up to 24 months, most preferably up to 36 months. Suspensions formed from the dry powder formulations are also stable for extended periods of time, and can be prepared by the pharmacist or patient prior to use. The suspensions should be stable for at least two weeks to 12 months, most preferably for at least 16 months.

The suspension formulations disclosed herein should exhibit reduced variability in bioactive agent content between batches such that bioactive agents can consistently be delivered in therapeutically effective amounts.

### SUMMARY OF THE INVENTION

Dry powder drug formulations of antitussives, antihistamines, decongestants and/or anticholinergics, tannic acid, dispersing agents, and viscosity modifying agents that have improved shelf-life are disclosed. As used herein, improved shelf-life means that the percent degradation of the one or more bioactive agents is less than 5%, less than 4%, less than 3%, less than 2%, or less than 1% after a period of at least two weeks, three weeks, four weeks, one month, six weeks, two months, three months, six months, twelve months, eighteen months, twenty-four months, thirty months, or thirty-six months.

Suspensions of the dry powder formulations in water, or other pharmaceutically acceptable solvent or cosolvents, also have improved shelf-life due to the inclusion of dispersing agents and viscosity modifying agents in the dry powder formulation. Suspensions formed from the dry powder formulations disclosed herein are stable over a period of time of at least two weeks, three weeks, four weeks, one month, six weeks, two months, three months, four months, six months, eight months, ten months, twelve months, or sixteen months.

In one embodiment the dry powder formulation contains phenylephrine hydrochloride, dextromethorphan hydrobromide, chlorpheniramine maleate, tannic acid, dispersants, and viscosity modifying agents. Suspensions can be formed by either a pharmacist or the patient prior to use.

### DETAILED DESCRIPTION OF THE INVENTION

### I. Definitions

“Antihistamine” as generally used herein refers to histamine H₁ receptor antagonists.

“Antitussive” as generally used herein refers to a cough suppressant that elevates the threshold for coughing.

“Bioactive agent” as generally used herein refers to pharmaceutical agents containing functional groups suitable for forming tannic salts. These require a basic functional group, such as an amine, to react with tannic acid and form a salt.

“Decongestant” as generally used herein refers to a compound which is an α-adrenergic agonist. Decongestants cause vasoconstriction in the mucosa of the respiratory tract which leads to decongestive properties.

“Degradation” as generally used herein refers to change or changes in the chemical structure, molecular formula, and/or molecular weight of the bioactive agent. Degradation can be an increase in mass as a result of reactions between one or more bioactive agents, other components, or combinations thereof. Alternatively, degradation can be a decrease in mass. Degradation of a bioactive agent can be determined by methods known to those skilled in the art. For example, high performance liquid chromatography (HPLC) can be coupled to a fluorescence or UV detector to determine peak purity. Degradation of bioactive agents by these methods will typically be detected as a broadened peak or the appearance of additional peaks, when compared with the trace of the parent bioactive agent. HPLC can also be coupled with mass spectrometry (HPLC/MS or HPLC/MS/MS) to determine sample purity and/or the degradation products. Degradation of bioactive agents will typically manifest as peaks and mass fragments corresponding to the degradation products that differ from the peak and/or mass of the parent bioactive agent.

“Dispersing agent” and “dispersant,” used interchangeably herein, generally refer to a chemical which improves separation of particles and prevents settling or clumping when introduced into a suspension.

“Dry powder” as generally used herein refers to solid granules which are essentially free of solvent, such as water. The dry powder can be the product of mixing constituent dry powders. The dry powder can also result from removing the solvent, drying, or otherwise dessicating any liquid solution, slurry, paste, or suspension containing solid components.

“Stable dry powder” as generally used herein refers to a dry powder formulation that does not degrade at 25° C. and 60% relative humidity over a shelf-life period of at least two weeks, three weeks, four weeks, one month, six weeks, two months, three months, six months, twelve months, eighteen months, twenty-four months, thirty months, or thirty-six months. For example, a dry powder is considered stable if the percent degradation of the one or more bioactive agents is less than 5%, less than 4%, less than 3%, less than 2%, or less than 1% as measured by high performance liquid chromatography (HPLC). Stable dry powders do not exhibit the following characteristics when compared to an equivalent freshly prepared dry powder formulation: visual discoloration, increase in moisture content greater than 5%, and/or increase in microbial content greater than 5%. Upon suspension, stable dry powder formulations should not exhibit sedimentation, decreased dispersability, increases or decreases in pH greater than 1.0, preferably 0.5, pH units, and flocculation, when compared to suspensions of an equivalent freshly prepared dry powder formulation.

“Pharmaceutically acceptable liquid” as generally used herein refers to one or more solvents, such as water or alcohol, used for processing or formulation of the dry powder formulation.

“Stable suspension” as generally used herein refers to a homogenous suspension of an agent or agents in a pharmaceutically acceptable liquid, preferably water, which remains uniformly dispersed for a period of at least 24, 30 hours, 36 hours, or 48 hours at 25° C. and 60% relative humidity. For example, a suspension is considered stable if the percent degradation of the one or more bioactive agents is less than 5-10%, preferably less than 5%, less than 4%, less than 3%, less than 2%, or less than 1% as measured by high performance liquid chromatography (HPLC). Stable suspensions also do not exhibit the following characteristics when compared to an equivalent freshly prepared suspension formulation: visual discoloration and increase in microbial content. Stable suspension formulations should not exhibit sedimentation, decreased dispersability, increases or decreases in pH greater than 1.0, preferably 0.5, pH units, and flocculation, when compared to freshly prepared suspensions of an equivalent dry powder formulation.

“Suspension” as generally used herein refers to a heterogeneous fluid containing solid particles sufficiently large for sedimentation.

“Tannate salt” as generally used herein refers to a complex of a basic compound and tannic acid.

“Tannic acid” as generally used herein refers to compounds with a central polyol core whose hydroxyl groups have been esterified with phenolic benzoic acid derivatives. The central polyol core is preferably glucose.

### II. Dry Powder Formulations

The dry powder formulations contain one or more bioactive agents, tannic acid, dispersants, viscosity modifying agents, and, optionally, one or more additional pharmaceutically acceptable excipients.

A. Bioactive Agents

Bioactive agents may be therapeutic, prophylactic, and/or diagnostic agents. The bioactive agents can be in the form of the free base or a pharmaceutically acceptable salt. Preferred counterions include bitartrate, maleate, citrate, chloride, bromide, fluoride, iodide, acetate, and sulfate. The following are preferred therapeutics.

1. Antihistamines

Antihistamines, classified as H₁ receptor antagonists, are used for the prophylaxis and relief of symptoms of hypersensitivity reactions such as allergic rhinitis, allergic conjunctivitis, urticaria, pruritus, sinusitis, angioedema, and anaphylaxis.

There are two types of antihistamines: first generation and second generation. The older antihistamines (first generation antihistamines) are associated with sedative and anti-muscarinic effects. These older antihistamines are distinguished from the newer (second generation) antihistamines which are essentially devoid of sedative effects.

In one embodiment, the antihistamine is selected from a non-exclusive list of first generation antihistamines including brompheniramine, chlorpheniramine, dexbrompheniramine, dexchlorpheniramine, carbinoxamine, clemastine, diphenhydramine, pyrilamine, tripelennamine, tripolidine, methdilazine, bromodiphenhydramine, promethazine, azatadine, cyproheptadine, diphenylpyraline, doxylamine, trimeprazine, phenindamine, hydroxyzine, ketotifen, tazifylline, meclazine, setastine, oxatomide, levocarbastine, lodoxamide, pheniramine, propiomazine, emedastine, flunarizine, meclozine, mefenidramine, methylsulfate and mepyramine.

In one embodiment, the antihistamine is in the free base form. In another embodiment, the first or second generation antihistamine is a salt.

In one embodiment, the antihistamine is present in a dry powder formulation from about 0.5% to about 10.0% w/w (% weight/weight), more preferably in an amount from about 0.5% to about 5.0% w/w, most preferably in an amount of about 1.5% w/w.

In one embodiment, the antihistamine is present in a suspension formulation from about 0.05% to about 5.00% w/v (% weight/volume), preferably in an amount from about 0.05% to about 0.2% w/v (% weight/volume), more preferably in an amount of about 0.12% w/v (% weight/volume).

In one embodiment, the antihistamine is the free base of chlorpheniramine, 3-(p-chlorophenyl)-3-(2-pyridyl)-N,N-dimethylpropylamine. In a preferred embodiment, the antihistamine is a salt of chlorpheniramine, preferably chlorpheniramine maleate. In one embodiment, the chlorpheniramine maleate is present in a dry powder formulation from about 0.5% to about 10% w/w (% weight/weight), more preferably from about 0.5% to about 5.0% w/w, most preferably about 1.5% w/w.

In another embodiment, chlorpheniramine maleate is present in a suspension formulation in an amount from about 0.05% to about 5.00% w/v (% weight/volume), preferably in an amount from about 0.05% to about 0.20% w/v (% weight/volume), more preferably in an amount of about 0.12% w/v (% weight/volume).

In another embodiment, the antihistamine is fexofenadine, loratadine, descarboethoxyloratadine, norastemizole, desmethylastemizole, cetirizine, acrivastine, ketotifen, temelastine, ebastine, epinastine, mizolastine, and setastine, astemizole, levocetirizine, rupatadine, mizolastin, noberastine or mequitazine.

2. Decongestants

Decongestants typically act on sympathomimetic targets, such as α-adrenergic receptors. The result is vasoconstriction which leads to decongestive properties.

In one embodiment, the decongestant is phenylephedrine, pseudoephedrine, levo-methamphetamine, naphazoline, oxymetazoline, phenylpropanolamine, propylhexedrine, Synephrine tetrahydrozoline, cyclopentamine, epinephrine, fenoxazoline, levonordefrin, mephentermine, metizoline, norepinephrine, tramazoline, tuaminoheptane, or tymazoline.

In one embodiment, the decongestant is the free base form.

In another embodiment, the decongestant is a salt.

In one embodiment, the decongestant is present in a dry powder formulation in an amount from about 1.0% to about 20.0% w/w (% weight/weight), more preferably in an amount from about 2.0% to about 10.0% w/w, most preferably in an amount of about 2.5% w/w.

In another embodiment, the decongestant is present in a suspension formulation in an amount from about 0.15% to about 5.00% w/v (% weight/volume), preferably in an amount from about 0.15% to about 2.00% w/v (% weight/volume), more preferably in an amount of about 0.20% w/v (% weight/volume).

In one embodiment, the decongestant is the free base of phenylephrine, (R)-(−)-3-Hydroxy-α-(methylaminomethyl)benzyl alcohol. In one embodiment, the decongestant is a salt of phenylephrine. In a preferred embodiment, the phenylephrine is present as the phenylephrine hydrochloride (HCl) salt.

In one embodiment, phenylephrine hydrochloride is present in a dry powder formulation in an amount from about 1.0% to about 20.0% w/w (% weight/weight), preferably in an amount from about 2.0% to about 10.0% w/w, more preferably in an amount of about 2.5% w/w.

In one embodiment, phenylephrine HCl is present in a suspension formulation in an amount from about 0.10% to about 5.00% w/v (% weight/volume), preferably in an amount from about 0.10% to about 2.00% w/v (% weight/volume), more preferably in an amount of about 0.20% w/v (% weight/volume).

3. Antitussives

Antitussives, or cough suppressants, are compounds that elevate the threshold for coughing. In one embodiment, the antitussive is carbetapentane, dextromethorphan, codeine, hydrocodone, oxycodone, or morphine.

In one embodiment, the antitussive is present as the free base form. In another embodiment, the antitussive is a salt.

In one embodiment, the antitussive is present in a dry powder formulation in an amount from about 1.0% to about 20.0% w/w (% weight/weight), preferably in an amount from about 2.0% to about 15.0% w/w, more preferably in an amount of about 3.8% w/w.

In one embodiment, the antitussive is present in a suspension from about 0.20% to about 5.00% w/v (% weight/volume), preferably in an amount from about 0.20% to about 3.00% w/v (% weight/volume), more preferably in an amount of about 0.30% w/v (% weight/volume).

In one embodiment, the antitussive is the free base of dextromethorphan, (9S,13S,14S)-3-Methoxy-17-methylmorphinan.

In another embodiment, the antitussive is a salt of dextromethorphan. In a most preferred embodiment, the antitussive is dextromethorphan hydrobromide (HBr).

In one embodiment, dextromethorphan hydrobromide is present in a dry powder formulation in an amount from about 1.0% to about 20.0% w/w (% weight/weight), preferably in an amount from about 2.0% to about 15.0% w/w, more preferably in an amount of about 3.8% w/w.

In one embodiment, dextromethorphan HBr is present in a suspension formulation in an amount from about 0.520% to about 5.00% w/v (% weight/volume), preferably in an amount from about 0.20% to about 3.00% w/v (% weight/volume), more preferably in an amount of about 0.30% w/v (% weight/volume).

4. Anticholinergics

Anticholiergics block the action of the neurotransmitter acethycholine and inhibit parasympathetic nerve impulses. Anticholinergics encompass two classes of antagonists: antimuscarinics and antinicotinics. Antimuscarinics operate on muscarinic receptors and primarily block post-ganglionic parasympathetic receptors. Antinicotinics operate on nicotinic receptors found in the autonomic ganglia and motor end plates in the parasympathetic system. Both classes affect involuntary movements of smooth muscle tissue and are useful in treating a variety of disorders including irritable bowel syndrome, asthma, bronchitis, Parkinson's disease, and bradycardia. Anticholinergics are also used as anaesthetics, antispasmodics, and mydriatic agents.

The acetylcholine receptor antagonists which be employed in the present formulations are known to those skilled in the art. U.S. Pat. Nos. 5,011,853 and 5,552,407 describe acetylcholine receptor antagonists. In one embodiment, the anticholinergic is selected from the list including, but not limited to, atropine, scopolamine, homatropine, atropine, methscopolamine, methylatropine, ipratropium, methylecgonidine (MEG), mecamylamine, benactyzine, benztropine, trihexyphenidyl, biperiden, procyclidine, benzetimide, dexetimide, dicycloverine, tolterodine, oxybutynin, pirenzepine, telenzepine, tiotropium, combinations thereof, and pharmaceutically acceptable derivatives thereof.

In one embodiment, the anticholinergic is present in a dry powder formulation in an amount from about 0.2% to about 3.0% w/w (% weight/weight), more preferably in an amount from about 0.3% to about 2.0% w/w, most preferably in an amount of about 0.35% w/w.

In one embodiment, the anticholinergic is present in a suspension in an amount from about 0.01% to about 0.3% w/v (% weight/volume), preferably in an amount from about 0.02% to about 0.2% w/v (% weight/volume), more preferably in an amount of about 0.025% w/v (% weight/volume).

In one embodiment, the anticholinergic is the free base of methscopolamine, 7(S)-(1α,2β,4β,5α,7β)-7-(3-Hydroxy-1-oxo-2-phenylpropoxy)-9,9-dimethyl-3-oxa-9-azoniatricyclo[3.3.1.0]nonane. In another embodiment, the anticholinergic is a salt of methscopolamine. In a preferred embodiment, the anticholinergic is methscopolamine nitrate.

In one embodiment, methscopolamine nitrate is present in a dry powder formulation in an amount from about 0.2% to about 3.0% w/w (% weight/weight), more preferably in an amount from about 0.3% to about 2.0% w/w, most preferably in an amount of about 0.35% w/w.

In one embodiment, methscopolamine nitrate is present in a suspension formulation in an amount from about 0.01% to about 0.3% w/v (% weight/volume), preferably in an amount from about 0.02% to about 2.0% w/v (% weight/volume), more preferably in an amount of about 0.025% w/v (% weight/volume).

B. Tannic Acid

Naturally occurring tannic acid contains a mixture of compounds produced from Turkish or Chinese nutgall that has a complex non-uniform chemistry. The compounds are considered to be secondary metabolites, and have a molecular weight between 500 Daltons and 5,000 Daltons. Tannic acid is a pale tan powder having a decomposition point of 210-215° C. and is highly soluble in water and alcohols. Tannins are structurally complex phenol-rich polymers which can be further divided into two classes: hydrolyzable tannins and condensed tannins.

Hydrolyzable tannins, or tannic acids, are composed of gallic acid or its condensation product, composed of ellagic acid esterified to the hydroxyl groups of a carbohydrate monomer. As the name implies, hydrolyzable tannins have ester bonds which are readily saponified under mildly acidic or basic conditions to give the parent carbohydrate and functionalized phenolic benzoic acids.

In one embodiment, D-glucose serves as the central component, with the hydroxyl groups esterified (or partially esterified) with functionalized phenolic benzoic acids. In a preferred embodiment, tannic acid contains at least three hydroxyl groups of the glucose monomer which have been esterified. In the most preferred embodiment, tannic acid contains all five hydroxyl groups of the glucose monomer which have been esterified. Tannic acid can be naturally or synthetically derived.

In one embodiment, tannic acid is present in a dry powder formulation in an amount from about 2.5% to about 50.0% w/w (% weight/weight), preferably in an amount from about 103.0% to about 30.0% w/w, more preferably in an amount of about 6.0% w/w.

In one embodiment, tannic acid is present in a suspension formulation in an amount from about 0.20% to about 10.0% w/v (% weight/volume), preferably in an amount from about 0.30% to about 5.00% w/v (% weight/volume), more preferably in an amount of about 0.5% w/v (% weight/volume).

C. Dispersants

The dispersant prevents clumping and aggregation of the tannate salt complex under aqueous or solvent conditions and promotes uniformity in the suspension upon addition of a pharmaceutically acceptable liquid.

In a preferred embodiment, dispersant or dispersants are present in an effective amount such that the suspension does not coagulate, agglomerate, or flocculate for at least two weeks, three weeks, four weeks, one month, six weeks, two months, three months, six months, twelve months, eighteen months, twenty-four months, thirty months, or thirty-six months, following suspension of the dry powder formulation. In addition, the quantity of dispersant or dispersants is present in an effective amount such that the suspension does not sediment or separate into multiple layers over a period of time of at least two weeks, three weeks, four weeks, two months, three months, four months, six months, eight months, ten months, twelve months, preferably sixteen months, following suspension of the dry powder formulation.

In one embodiment, the dispersant is a carrageenan compound. Carrageenans are a family of linear sulfated polysaccharides of D-galactose and 3,6-anhydro D-galactose which are extracted from red seaweeds. Members of the carrageenan family, including kappa, iota, and lambda carrageenans, are distinguished by the position and number of sulfate groups present. Iota carageenan is available under the tradename Seaspen PF by FMC Biopolymer, Philadelphia, Pa.

In another embodiment, the dispersant is maltodextrin. Maltodextrin is a polysaccharide of D-glucose connected by α-linkages, generally three to nineteen glucose units in length, generated from partial hydrolysis of starch.

In yet another embodiment, a combination of dispersants is used. In a particular embodiment, a combination of carrageenan and maltodextrin is used.

In other particular embodiments, the one or more dispersants does not contain magnesium aluminum silicate. In other particular embodiments, the one or more dispersants does not contain magnesium aluminum silicate and/or xantham gum.

In one embodiment, the dispersant(s) is present in a dry powder formulation in an amount from about 5.0% to about 50.0% w/w (% weight/weight), preferably in an amount from about 10.0% to about 40.0% w/w, more preferably in an amount of about 35.0% w/w.

In one embodiment, the dispersant or dispersants are present in a suspension formulation in an amount from about 0.50% to about 10.0% w/v (% weight/volume), preferably in an amount from about 0.75% w/v to about 35.00% w/v. In some preferred embodiments, the dispersant or dispersants are present in a suspension formulation in an amount between 0.50%-1.0%, 0.50%-1.5%, 0.50%-2.0%, 0.50%-2.5%, 0.50%-3.0%, or 0.50%-5.0% w/v. In the most preferred embodiment, the dispersant or dispersants are present in the suspension formulation in an amount of about 3.0% (% weight/volume).

D. Viscosity Modifying Agents

In preferred embodiments, the formulations include a viscosity modifying agent which stabilizes the suspension. In the most preferred embodiment, the viscosity modifying agent is propylene glycol alginate. Propylene glycol alginate is an ester product of the reaction between propylene oxide and alginic acid, in which some of the carboxyl groups on alginic acid are esterified with propylene glycol. The remaining carboxyl groups can be neutralized with an appropriate alkali and/or remain free carboxyl groups. Propylene glycol alginate is also known as 1,2-propane-diol ester of alginic acid, hydroxypropyl alginate, and propane 1,2-diol alginate. Propylene glycol alginate is sold under the tradename Protanal® Ester SD-LB, which is available from FMC Biopolymer, Philadelphia, Pa.

Alginates are polyuronic acids which are major components of the cell walls of brown seaweed. Alginates have valuable rheological properties which can be varied to a great extent by varying the degree of polymerization of the polysaccharide and the ionic environment. Alginates can provide suspensions having a range of viscosities and colloidal properties for various pharmaceutical applications.

In one embodiment, the viscosity modifying agent(s) is present in a dry powder formulation in an amount from about 3.0% to about 50.0% w/w (% weight/weight), preferably in an amount from about 5.0% to about 40.0% w/w, more preferably in an amount of about 25.0% w/w.

In one embodiment, the viscosity modifying agent or agents are present in a suspension formulation in an amount from about 0.50% w/v to about 10.0% w/v (% weight/volume), preferably in an amount from about 0.75% w/v to about 5.00% w/v. In some preferred embodiments, the viscosity modifying agent or agents are present in a suspension formulation between 0.50%-1.0%, 0.50%-1.5%, 0.50%-2.0%, 0.50%-2.5%, or 0.50%-3.0% w/v. In the most preferred embodiment, the viscosity modifying agent or agents are present in the suspension formulation in an amount of about 2.00% (% weight/volume).

In a preferred embodiment, one or more viscosity modifying agents are present in an effective amount such that the suspension exhibits optimal viscosity properties for at least two weeks, three weeks, four weeks, one month, six weeks, two months, three months, four months, six months, eight months, ten months, twelve months, preferably sixteen months, following suspension of the dry powder formulation. In one embodiment, the optimal viscosity of the suspension is between 1000 cPs and 7000 cPs, more preferably between 2000 cPs and 5000 cPs, and most preferably between 2000 cPs and 4000 cPs.

The viscosity and physical appearance of suspension formulations are influenced by the ratio of both dispersant (such as carrageenan) and viscosity modifying agent (such as propylene glycol alginate). The relative concentration of both components affect the ability of a suspension to resist separation over time and display the optimal viscosity properties, namely between 1000 cPs and 7000 cPs, more preferably between 2000 cPs and 5000 cPs, and most preferably between 2000 cPs and 4000 cPs.

Keeping the amount of viscosity modifying agent constant, suspensions that contain one or more dispersants in a concentration that is too low will result in separation. Suspensions containing one or more dispersants in a concentration that is too high will result in a suspension that has a heightened amount of solids, such that shaking does not give a dispersed suspension.

Keeping the amount of dispersant constant, suspensions that contain one or more viscosity modifying agents in a concentration that is too low will not have the desired viscosity characteristics, namely a viscosity above 2000 cPs. Suspensions containing one or more viscosity modifying agents in a concentration that is too high will have viscosities greater than 4000 cPs.

In one embodiment, the ratio of dispersant to viscosity modifying agent in a suspension formulation is about 0.5%-5.0% (% w/v) dispersant to about 1.0%-3.0% (% w/v) viscosity modifying agent. In a more preferred embodiment, the ratio is about 0.5%-3.0% (% w/v) dispersant to about 2% (% w/v) viscosity modifying agent. In the most preferred embodiment, the ratio is about 2.0% dispersant (% w/v) to about 3.0% (% w/v) viscosity modifying agent. In the preferred embodiments, the dispersant is carrageenan (Seaspen PF) and the viscosity modifying agent is propylene glycol alginate (Protanal Ester SD-LB).

E. Dry Powder Excipients

Dry powder formulations may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, plasticizers, pigments, colorants, stabilizing agents, glidants, surfactants, humectants, plasticizers, crystallization inhibitors, wetting agents, bulk filling agents, solubilizers, bioavailability enhancers, pH adjusting agents, celluloses, crystallization inhibitors, wetting agents, and combinations thereof.

Examples of suitable lubricants include, but are not limited to, magnesium stearate, calcium stearate, stearic acid, glycerol behenate, polyethylene glycol, talc, and mineral oil.

Disintegrants facilitate dosage form disintegration, or “breakup,” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, and ammonium salts of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The powder may also contain nontoxic auxiliary substances such as dyes, pH buffering agents, preservatives, and sweeteners.

The most preferred excipients and their respective amounts, when present in a dry powder, are: sodium alginate (Seaspen PF), 15-25% (% w/w); silicon dioxide (Cab-o-sil 5-MP), 0.5%-2.5% (% w/v); maltodextrin (Maltrin QD M585), 10.0-20.0% (% w/w); polyvinylpyrrolidone (Povidone K30), 2-10% (% w/w); polyethylene glycol (PEG 3350), 2-10% (% w/w); Sucralose, 0.5-4.0% (% w/w); sodium benzoate, 0.5-4.0% (% w/w); citric acid, 0.5-4% (% w/w); sodium citrate, 0.5-3.0% (% w/w); and dye, 0.03-1.5% (% w/w).

The most preferred excipients and their respective amounts, when suspended in a pharmaceutically acceptable liquid, most preferably water, are: sodium alginate (Seaspen PF), 1.0%-5.0% (% w/v); silicon dioxide (Cab-o-sil M-5P), 0.05%-1.0% (% w/v); maltodextrin (Maltrin QD M585), 0.50%-3.0% (% w/v); polyvinylpyrrolidone (Povidone K30), 0.10%-1.0% (% w/v); polyethylene glycol (PEG 3350), 0.10%-1.0% (% w/v); Sucralose, 0.05%-0.60% (% w/v); sodium benzoate, 0.05%-0.30% (% w/v); citric acid, 0.05%-0.60% (% w/v); sodium citrate, 0.05%-0.30% (% w/v); and dye, about 0.01%.

### III. Methods of Preparing Dry Powder Formulations

A. Formation of the Dry Powder for Storage

The tannic acid, one or more bioactive agents, one or more dispersing agents, and one or more viscosity modifying agents are mixed in their dry, non-reactive states to obtain a uniform blend. Optionally, any solid excipients can also be added at this point and concurrently mixed, to form a uniform blend. The bioactive agent should be in a stable form, such as the free base or acid. The tannic acid:drug ratio is from about 0.5:1 to about 3:1 ratio to obtain proper tannate salt conversion upon suspension of the dry powder.

The ingredients are mixed with a suitable powder blender with low shear. Such blenders are well known in the art and include, but are not limited to, V-blenders, paddle blenders, planetary blenders, or ribbon blenders. The ingredients are blended for from about 5 minutes to about 30 minutes.

Next, a container is filled with the dry powder for storage. Suitable containers include, but are not limited to, 1 oz., 4 oz., 8 oz., or 16 oz. bottles. Other suitable containers include sealed bags. In a preferred embodiment, the container is suitable for storage of the dry powder such that the powder is stable for at least 2 weeks, more preferably at least 1 month, more preferably six weeks, more preferably at least 2 months, more preferably at least 3 months, more preferably at least 4 months, more preferably at least 5 months, more preferably at least 6 months, more preferably at least 7 months, more preferably at least 8 months, more preferably at least 9 months, more preferably at least 10 months, more preferably at least 11 months, more preferably a least 12 months, more preferably up to 24 months, most preferably up to 36 months prior to suspension.

Stability can be measured by methods known to those skilled in the art including moisture content, microbial content, appearance, color, dispersability, pH, and purity. The percent degradation of the one or more bioactive agents of the dry powder formulation should be less than 5% at 25° C. and 60% relative humidity and/or 40° C. and 75% relative humidity in long-term storage. In a preferred embodiment, the percent degradation of the one or more bioactive agents is less than 5%, less than 4%, less than 3%, less than 2%, or less than 1% as measured by high performance liquid chromatography (HPLC).

In another embodiment, the stored dry powder does not significantly change in original color compared to a freshly prepared dry powder formulation, as determined by UV spectroscopy or visual observation. In a preferred embodiment, the stored dry powder is not brown in color.

In another embodiment, moisture content of the stored dry powder does not increase more than 5%, more than 4%, more than 3%, more than 2%, or more than 1%.

In another embodiment, microbial content of the stored dry powder does not increase. In another embodiment, the pH of a suspension of the stored dry powder does not differ more than 1.0, preferably 0.5, pH units from that of a suspension of freshly prepared dry powder.

In another embodiment, a suspension of stored dry powder does not contain solidified globular material formed by flocculation, agglomeration, or coagulation, but instead consists of finely dispersed particulate material. The presence of globular material can be determined visually or by methods known to those skilled in the art including multiple light scattering coupled with vertical scanning. In one embodiment, the dry powder does not form floccules, agglomerates, or other solidified globular material.

B. Formation of Suspensions

A suspension of the dry powder can be prepared by the pharmacist, prescribing physician, or patient prior to use. To avoid difficulties associated with degradation and impurities, it would be beneficial to delay formation of a suspension dosage as long as possible. The dry powder will be present in the container in an amount which will yield the appropriate dosage (mg/mL) when mixed with either 15 mL (1 tablespoon), 4 oz, 8 oz., or 16 oz. of a solvent, such as water, and shaken well for about 10 to about 30 seconds. This step converts the bioactive agents to their tannate salt form without clumping.

In preferred embodiments, the suspension is stable, and can be used over a period of time of at least two weeks, three weeks, four weeks, two months, three months, four months, six months, eight months, ten months, twelve months, preferably sixteen months. Stability can be measured by methods known to those skilled in the art including moisture content, microbial content, appearance, color, dispersability, pH, and purity.

The percent degradation of the one or more bioactive agents of the suspension formulation should not degrade more than 5% at 25° C. and 60% relative humidity and/or 40° C. and 75% relative humidity in long-term storage. In a preferred embodiment, the percent degradation of the one or more bioactive agents is less than 5%, less than 4%, less than 3%, less than 2%, or less than 1% as determined by high performance liquid chromatography (HPLC).

In one embodiment, the stable suspension does not visually separate to form two or more layers. In another embodiment, the stable suspension does not have sediment particulates at the bottom of the container. In another embodiment, the stable suspension does not significantly change in original color compared to a freshly prepared suspension, as determined by UV spectroscopy or visual observation. In a preferred embodiment, the stable suspension is not brown in color.

In another embodiment, moisture content does not increase by more than 5%, more than 4%, more than 3%, more than %, or more than 1%. In another embodiment, microbial content does not increase. In another embodiment, the pH of the suspension does not differ more than 1.0, preferably 0.5, pH units from that of a freshly prepared suspension.

In another embodiment, the stable suspension does not contain solidified globular material formed by flocculation, agglomeration, or coagulation. The presence of globular material can be determined visually or by methods known to those skilled in the art including multiple light scattering coupled with vertical scanning. In one embodiment, the suspension does not form floccules, agglomerates, or other solidified globular material.

The following examples are included to demonstrate preferred embodiments of the invention. It will be apparent to those of skill in the art that variations may be applied to the compositions and methods described herein without departing from the concept, spirit, and scope of the invention.

### EXAMPLES

### Example 1

### Suspension of a Dry Powder Formulation (1)

A 118 mL suspension was prepared with the following:

| | Percent | |
|---|---|---|
| | weight/volume | |
| Ingredient | (% w/v) | Amount (g) |
| Chlorpheniramine | 0.12 | 0.142 |
| maleate | | |
| Phenylephrine HCl | 0.20 | 0.236 |
| Dextromethorphan HBr | 0.30 | 0.354 |
| Tannic Acid | 0.47% | 0.549 |
| Seaspen PF | 1.50% | 1.770 |
| Cab-o-sil M-5P | 0.15% | 0.177 |
| Maltrin QD M585 | 1.50% | 1.770 |
| Povidone K30 | 0.50% | 0.590 |
| PEG 3350 | 0.50% | 0.590 |
| Protanal Ester SD-LB | 2.00% | 2.360 |
| Sucralose | 0.20% | 0.236 |
| Sodium Benzoate | 0.15% | 0.177 |
| Citric Acid | 0.20% | 0.236 |
| Sodium Citrate | 0.10% | 0.118 |
| Dye | 0.01% | 0.012 |
| Purified Water | 92.11% | 108.684 |
| Total | 100 | 118.000 |

### Example 2

### Suspension of a Dry Powder Formulation (2)

A 473 mL suspension was prepared with the following:

| | Percent | |
|---|---|---|
| | weight/volume | |
| Ingredient | (% w/v) | Amount (g) |
| Chlorpheniramine | 0.12% | 0.568 |
| maleate | | |
| Phenyleprine HCl | 0.20% | 0.946 |
| Dextromethorphan HBr | 0.30% | 1.419 |
| Tannic Acid | 0.47% | 2.199 |
| Seaspen PF | 1.50% | 7.095 |
| Cab-o-sil M-5P | 0.15% | 0.710 |
| Maltrin QD M585 | 1.50% | 7.095 |
| Povidone K30 | 0.50% | 2.365 |
| PEG 3350 | 0.50% | 2.365 |
| Protanal Ester SD-LB | 2.00% | 9.460 |
| Sucralose | 0.20% | 0.946 |
| Sodium Benzoate | 0.15% | 0.710 |
| Citric Acid | 0.20% | 0.946 |
| Sodium Citrate | 0.10% | 0.473 |
| Dye | 0.01% | 0.047 |
| Purified Water | 92.11% | 435.657 |
| Total | 100 | 473.000 |

### Example 3

### Effect of Varying the Weight Percentages of Dispersant and Viscosity Modifying Agent on Viscosity

Suspension formulations with varying amounts of propylene glycol alginate (viscosity modifying agent) and carageenan (dispersant) in 118 mL of water were prepared with the agents below. The viscosities of the suspensions were determined at various time points shown below.

| | Percent | | | | | | |
|---|---|---|---|---|---|---|---|
| | weight/volume | Percent | | | | | |
| | (% w/v) | weight/volume | | | | | |
| | Propylene | (% w/v) | | | | | |
| | Glycol | Seaspen PF | Viscosity (cPs) | | | | |
| Sample | Alginate | (carrageenan) | Initial | 24 h | 48 h | 1 wks | 2 wks |
| 1 | 2.0 | 2.5 | 4524 | 4132 | 3889 | 4008 | 4127 |
| 2 | 3.0 | 0.5 | 9477 | 7204 | 7382 | 7461 | 6429 |
| 3 | 1.0 | 2.5 | 635 | 595 | 555 | 427 | 555 |
| 4 | 2.0 | 0.5 | 3175 | 3016 | 3135 | 2738 | 3016 |
| 5 | 2.0 | 1.5 | 3889 | 3135 | 2976 | 3175 | 2103 |
| 6 | 1.0 | 1.5 | 873 | 397 | 317 | 595 | 515 |
| 7 | 3.0 | 1.5 | 14320 | 14090 | 13850 | 12660 | 11430 |
| 8 | 1.0 | 0.5 | 2738 | 833 | 1111 | 635 | 595 |
| 9 | 3.0 | 2.5 | 17500 | 10550 | 12220 | 14800 | 15200 |

### Example 4

### Physical Characteristics of Dry Powder Suspension Stability

The physical characteristics of the suspension formulations in Example 3 were visually observed and measured at various time points. The results are shown below.

| | Physical Characteristics | | | | |
|---|---|---|---|---|---|
| Sample | Initial | 24 h | 48 h | 1 wk | 2 wks |
| 1 | uniform^{a} | uniform | uniform | Uniform | uniform |
| | after | | | | |
| | scraping^{b,e} | | | | |
| 2 | uniform | uniform | uniform | Uniform | uniform |
| 3 | uniform | uniform | uniform | uniform | uniform |
| 4 | uniform | uniform | uniform | uniform | uniform |
| 5 | uniform | uniform | uniform | uniform | uniform |
| 6 | uniform | separation^{c} | separation | separation | separation |
| 7 | uniform | uniform | uniform | uniform | uniform |
| | after | | | | |
| | scraping^{e} | | | | |
| 8 | uniform | separation | separation^{d} | separation | separation |
| 9 | uniform | uniform | uniform | uniform | uniform |
| | after | | | | |
| | scraping^{e} | | | | |

^{a}uniform suspension
^{b}spatula required to remove dried powder from bottom of container
^{c}two layers of at least 1 mm thickness visually observed
^{d}three layers of at least 1 mm thickness visually observed
^{e}water had to be added in two portions to generate suspension

Discussion

Examples 3 and 4 illustrate that the viscosity and physical appearance of suspension formulations are affected by the amount of dispersant (carrageenan) and viscosity modifying agent (propylene glycol alginate). Samples 1, 7, and 9, contained the two highest concentrations of propylene glycol alginate (2.0% or 3.0% w/v) and the highest concentration of carrageenan (2.5% w/v). These samples contained elevated amounts of solids that could not be dispersed by shaking only. Both manual agitation with a spatula and step-wise water addition were required to form the suspension. In addition, suspensions 7 and 9 exhibited higher than desired viscosities due to the heightened concentration of propylene glycol alginate present. Samples 6 and 8 contained the lowest concentration of propylene glycol alginate (1.0% w/v) and the two lowest concentrations of carrageenan (0.5% and 1.5% w/v). However, both of these suspension samples separated upon standing. Sample 2 remained uniform, even in the presence of the 3.0% w/v propylene glycol alginate. However, the viscosity was consistently greater than 6000 cPs. Sample 3 also remained uniform throughout the experiment. However, the viscosity was markedly lower than desired, consistently below 1000. Samples 4 and 5, both made with mid-range amounts of both propylene glycol alginate and carrageenan, exhibited optimal visual characteristics and viscosity. Neither suspension separated up to a period of 2 weeks. In addition, samples 4 and 5 had optimal viscosities of between 2000 cPs and 4000 cPs.

I claim:

1. A dry powder formulation comprising one or more bioactive agents, tannic acid, a high molecular weight polymeric dispersant, and a viscosity modifying agent,
wherein the dry powder formulation is stable for at least two weeks at 25° C. and 60% relative humidity.

2. A suspension formulation formed from the dry powder formulation of claim 1 and a pharmaceutically acceptable aqueous liquid,
wherein the suspension is stable for at least two weeks following suspension at 25° C. and 60% relative humidity.

3. The formulation of claim 1, wherein the bioactive agent is selected from the group consisting of antihistamines, decongestants, antitussives, anticholinergics, and combinations thereof.

4. The formulation of claim 2, wherein the bioactive agent is selected from the group consisting of antihistamines, decongestants, antitussives, anticholinergics, and combinations thereof.

5. The formulation of claim 3, wherein the antihistamine is present in an amount from about 0.05% to about 5.00% w/v (% weight/volume) upon suspension in a pharmaceutically acceptable aqueous liquid.

6. The formulation of claim 4, wherein the antihistamine is present in an amount from about 0.20% to about 5.00% w/v (% weight/volume) of the suspension.

7. The formulation of claim 5, wherein the antihistamine is present in about 0.12% w/v (% weight/volume) upon suspension in a pharmaceutically acceptable aqueous liquid.

8. The formulation of claim 6, wherein the antihistamine is present in about 0.12% w/v (% weight/volume) of the suspension.

9. The formulation of claim 3 wherein the antihistamine is selected from the group consisting of brompheniramine, chlorpheniramine, dexbrompheniramine, dexchlorpheniramine, carbinoxamine, clemastine, diphenhydramine, pyrilamine, tripelennamine, tripolidine, methdilazine, bromodiphenhydramine, promethazine, azatadine, cyproheptadine, diphenylpyraline, doxylamine, trimeprazine, phenindamine, hydroxyzine, ketotifen, tazifylline, meclazine, setastine, oxatomide, levocarbastine, lodoxamide, pheniramine, propiomazine, emedastine, flunarizine, meclozine, mefenidramine, methylsulfate, mepyramine, combinations thereof, and pharmaceutically acceptable salts thereof.

10. The formulation of claim 4 wherein the antihistamine is selected from the group consisting of brompheniramine, chlorpheniramine, dexbrompheniramine, dexchlorpheniramine, carbinoxamine, clemastine, diphenhydramine, pyrilamine, tripelennamine, tripolidine, methdilazine, bromodiphenhydramine, promethazine, azatadine, cyproheptadine, diphenylpyraline, doxylamine, trimeprazine, phenindamine, hydroxyzine, ketotifen, tazifylline, meclazine, setastine, oxatomide, levocarbastine, lodoxamide, pheniramine, propiomazine, emedastine, flunarizine, meclozine, mefenidramine, methylsulfate, mepyramine, combinations thereof, and pharmaceutically acceptable salts thereof.

11. The formulation of claim 3, wherein the antihistamine is selected from the group consisting of fexofenadine, loratadine, descarboethoxyloratadine, norastemizole, desmethylastemizole, cetirizine, acrivastine, ketotifen, temelastine, ebastine, epinastine, mizolastine, and setastine, astemizole, levocetirizine, rupatadine, mizolastin, noberastine, mequitazine, combinations thereof, and pharmaceutically acceptable salts thereof.

12. The formulation of claim 4, wherein the antihistamine is selected from the group consisting of fexofenadine, loratadine, descarboethoxyloratadine, norastemizole, desmethylastemizole, cetirizine, acrivastine, ketotifen, temelastine, ebastine, epinastine, mizolastine, and setastine, astemizole, levocetirizine, rupatadine, mizolastin, noberastine, mequitazine, combinations thereof, and pharmaceutically acceptable salts thereof.

13. The formulation of claim 11, wherein the antihistamine is chlorpheniramine maleate.

14. The formulation of claim 12, wherein the antihistamine is chlorpheniramine maleate.

15. The formulation of claim 3, wherein the decongestant is present in an amount from about 0.10% to about 5.00% w/v (% weight/volume) upon suspension in a pharmaceutically acceptable aqueous liquid.

16. The formulation of claim 4, wherein the decongestant is present in an amount from about 0.10% to about 5.00% w/v (% weight/volume) of the suspension.

17. The formulation of claim 15, wherein the decongestant is present at a concentration of about 0.20% w/v (% weight/volume) upon suspension in a pharmaceutically acceptable aqueous liquid.

18. The formulation of claim 16, wherein the decongestant is present at a concentration of about 0.20% w/v (% weight/volume) of the suspension.

19. The formulation of claim 15, wherein the decongestant is selected from the group consisting of phenylephedrine, pseudoephedrine, levo-methamphetamine, naphazoline, oxymetazoline, phenylpropanolamine, propylhexedrine, synephrine tetrahydrozoline, cyclopentamine, epinephrine, fenoxazoline, levonordefrin, mephentermine, metizoline, norepinephrine, tramazoline, tuaminoheptane, tymazoline, combinations thereof, and pharmaceutically acceptable salts thereof.

20. The formulation of claim 16, wherein the decongestant is selected from the group consisting of phenylephedrine, pseudoephedrine, levo-methamphetamine, naphazoline, oxymetazoline, phenylpropanolamine, propylhexedrine, synephrine tetrahydrozoline, cyclopentamine, epinephrine, fenoxazoline, levonordefrin, mephentermine, metizoline, norepinephrine, tramazoline, tuaminoheptane, tymazoline, combinations thereof, and pharmaceutically acceptable salts thereof.

21. The formulation of claim 19, wherein the decongestant is phenylephrine hydrochloride.

22. The formulation of claim 20, wherein the decongestant is phenylephrine hydrochloride.

23. The formulation of claim 3, wherein the antitussive is present in an amount from about 0.25% to about 5.00% w/v (% weight/volume) upon suspension in a pharmaceutically acceptable aqueous liquid.

24. The formulation of claim 4, wherein the antitussive is present in an amount from about 0.25% to about 5.00% w/v (% weight/volume) of the suspension.

25. The formulation of claim 23, wherein the antitussive is present in about 0.3% w/v (% weight/volume), upon suspension in a pharmaceutically acceptable aqueous liquid.

26. The formulation of claim 24, wherein the antitussive is present in about 0.3% w/v (% weight/volume) of the suspension.

27. The formulation of claim 23, wherein the formulation comprises an antitussive selected from the group consisting of carbetapentane, dextromethorphan, codeine, hydrocodone, oxycodone, morphine, combinations thereof, and pharmaceutically acceptable salts thereof.

28. The formulation of claim 24, wherein the formulation comprises an antitussive selected from the group consisting of carbetapentane, dextromethorphan, codeine, hydrocodone, oxycodone, morphine, combinations thereof, and pharmaceutically acceptable salts thereof.

29. The formulation of claim 27, wherein the antitussive is dextromethorphan hydrobromide.

30. The formulation of claim 28, wherein the antitussive is dextromethorphan hydrobromide.

31. The formulation of claim 3, wherein the anticholinergic is present in an amount from about 0.01% to about 0.3% w/v (% weight/volume) upon suspension in a pharmaceutically acceptable aqueous liquid.

32. The formulation of claim 4, wherein the anticholinergic is present in an amount from about 0.01% to about 0.3% w/v (% weight/volume) of the suspension.

33. The formulation of claim 31, wherein the anticholinergic is present in an amount of about 1.50% w/v (% weight/volume) upon suspension in a pharmaceutically acceptable aqueous liquid.

34. The formulation of claim 32, wherein the anticholinergic is present in an amount of about 1.50% w/v (% weight/volume) upon suspension in a pharmaceutically acceptable aqueous liquid.

35. The formulation of claim 31, wherein the formulation comprises an anticholinergic selected from the group consisting of atropine, scopolamine, homatropine, atropine, methscopolamine, hyoscyamine, methylatropine, ipratropium, methylecgonidine (MEG), mecamylamine, benactyzine, benztropine, trihexyphenidyl, biperiden, procyclidine, benzetimide, dexetimide, dicycloverine, tolterodine, oxybutynin, pirenzepine, telenzepine, tiotropium, clidinium, combinations thereof, and pharmaceutically acceptable salts thereof.

36. The formulation of claim 32, wherein the formulation comprises an anticholinergic selected from the group consisting of atropine, scopolamine, homatropine, atropine, methscopolamine, methylatropine, hyoscyamine, ipratropium, methylecgonidine (MEG), mecamylamine, benactyzine, benztropine, trihexyphenidyl, biperiden, procyclidine, benzetimide, dexetimide, dicycloverine, tolterodine, oxybutynin, pirenzepine, telenzepine, tiotropium, clidinium, combinations thereof, and pharmaceutically acceptable salts thereof.

37. The formulation of claim 35, wherein the anticholinergic is methscopolamine nitrate.

38. The formulation of claim 36, wherein the anticholinergic is methscopolamine nitrate.

39. The formulation of claim 1, wherein the concentration of tannic acid is from about 0.3% to about 5.00% w/v (% weight/volume) upon suspension in a pharmaceutically acceptable aqueous liquid.

40. The formulation of claim 2, wherein the concentration of tannic acid is from about 0.3% to about 5.00% w/v (% weight/volume) of the suspension.

41. The formulation of claim 1, wherein the dispersant is present in an amount from about 0.50% to about 10.0% w/v (% weight/volume) upon suspension in a pharmaceutically acceptable aqueous liquid.

42. The formulation of claim 2, wherein the dispersant is present in an amount from about 0.50% to about 10.0% w/v (% weight/volume) of the suspension.

43. The formulation of claim 41, wherein the dispersant is present in an amount from about 0.50% to 3.0%.

44. The formulation of claim 42, wherein the dispersant is present in an amount from about 0.50% to 3.0%.

45. The formulation of claim 1, wherein the dispersant is carrageenan or maltodextrin.

46. The formulation of claim 1, wherein the viscosity modifying agent is present in an amount from about 0.50% to about 10.0% w/v (% weight/volume) upon suspension in a pharmaceutically acceptable aqueous liquid.

47. The formulation of claim 2, wherein the viscosity modifying agent is present in an amount from about 0.50% to about 10.0% w/v (% weight/volume) of the suspension.

48. The formulation of claim 46, wherein the viscosity modifying agent is present in an amount from about 0.5% to about 2.0% w/v (% weight/volume).

49. The formulation of claim 47, wherein the viscosity modifying agent is present in an amount from about 0.5% to about 2.0% w/v (% weight/volume).

50. The formulation of claim 1, wherein the viscosity modifying agent is propylene glycol alginate.

51. The formulation of claim 1 further comprising dry powder excipients selected from the group consisting of diluents, binders, lubricants, disintegrators, fillers, plasticizers, pigments, colorants, stabilizing agents, glidants, surfactants, humectants, plasticizers, crystallization inhibitors, wetting agents, bulk filling agents, solubilizers, bioavailability enhancers, pH adjusting agents, and combinations thereof.

52. The formulation of claim 2 further comprising dry powder excipients selected from the group consisting of diluents, binders, lubricants, disintegrators, fillers, plasticizers, pigments, colorants, stabilizing agents, glidants, surfactants, humectants, plasticizers, crystallization inhibitors, wetting agents, bulk filling agents, solubilizers, bioavailability enhancers, pH adjusting agents, and combinations thereof.

53. The formulation of claim 2, wherein the viscosity of the suspension is between about 2000 cPs and about 4000 cPs.

54. The formulation of claim 1, wherein the one or more bioactive agents present in the dry powder formulation degrades less than about 5%, as determined by high performance liquid chromatography, for at least two weeks at 25° C. and 60% relative humidity for at least 36 months.

55. The formulation of claim 2, wherein the one or more bioactive agents present in the suspension formulation degrades less than about 5%, as determined by high performance liquid chromatography, for at least two weeks at 25° C. and 60% relative humidity for at least 16 months.

56. A method for the preparation of stable dry powder formulations, comprising:
(a) mixing one or more bioactive agents, tannic acid, a high molecular weight polymeric dispersant, and a viscosity modifying agent, all in their non-reactive dry powder forms, with a low shear blender until uniformly mixed, and
(b) filling a container with the dry powder,
wherein the one or more bioactive agents present in the dry powder formulation degrades less than about 5%, as determined by high performance liquid chromatography, for at least two weeks at 25° C. and 60% relative humidity.

57. A method for the preparation of a stable suspension of the dry powder formulation of claim 51, further comprising:
(c) the addition of a pharmaceutically acceptable aqueous liquid to the container,
wherein the one or more bioactive agents present in the suspension formulation degrades less than about 5%, as measured by high performance liquid chromatography, for at least two weeks at 25° C. and 60% relative humidity.

58. The formulation of claim 2, wherein ratio of the dispersant and viscosity modifying agent is about 3.0% dispersant (% w/v) to about 2.0% (% w/v) viscosity modifying agent.
